(19) United States
(12) Patent Application Publication
(10) Pub. No.: US 2023/0218522 A1
(43) Pub. Date: Jul. 13, 2023

(54) USES OF SUPER-OXYGENATED WATER AND GEL

(71) Applicant: GIS Ventures Inc., Saint Andrews (CA)

(72) Inventor: Michael Beattie, Saint Andrews (CA)

(73) Assignee: GIS Ventures Inc., Saint Andrews, NB (CA)

(21) Appl. No.: 17/996,259

(22) PCT Filed: Apr. 26, 2021
(86) PCT No.: PCT/CA2021/050569

§ 371 Date: Oct. 14, 2022

Related U.S. Application Data
(60) Provisional application No. 63/014,764, filed on Apr. 24, 2020.

(51) Int. Cl. A61K 9/08; A61K 33/00; A61K 9/06

(52) U.S. Cl. CPC A61K 9/08; A61K 33/00; A61K 9/06

(57) ABSTRACT

A super-oxygenated fluid selected from water, an aqueous solution and a gel is introduced to a living organism via any one of the following routes: transcutaneous, intradermal, subcutaneous, intraperitoneal and oral.

### FIELD

This invention relates to the use of oxygen by biologicals, including humans.

### BACKGROUND

It is well known that in living organisms, molecular oxygen has a key role in cellular health. A detailed description of the benefits of molecular oxygen is found in U.S. Provisional Patent Application Ser. No. 63/014,764, filed Apr. 24, 2020, incorporated herein by reference.

Hyperbaric chambers, which rely on the fact that oxygen can be absorbed through the skin, are commonly used as a means of maximizing the body's access to oxygen. For example, hyperbaric chambers are commonly used by athletes to aid in recovery from high-exertion events.

However, use of a hyperbaric chamber for extended periods of time is both impractical and medically dangerous. Specifically, elevated blood-oxygen levels severely hamper the proper exchange of blood-oxygen with CO₂ released from cells. This, in turn, releases a cascade of secondary issues mainly involving the Haldane and Bohr Effects.

### SUMMARY

According to an aspect of the invention, a super-oxygenated fluid selected from water, an aqueous solution and a gel, is introduced to a living organism via any one of the following routes: transcutaneous, intradermal, subcutaneous, intraperitoneal and oral.

According to another aspect of the invention, in the fluid, the level of dissolved oxygen can be greater than 185% of saturation (i.e. 185% DO) at normal total gas pressure (TGP).

According to another aspect of the invention, the fluid can be oxygenated using the device described in U.S. Pat. No. 6,209,855.

According to another aspect, the fluid can be used in the healing of a wound, wound pO₂ can be monitored in real-time and the fluid can be introduced in response to wound site pO₂.

### DETAILED DESCRIPTION OF EMBODIMENTS OF THE INVENTION

One embodiment of the invention involves the transcutaneous introduction of the fluid to an organism for accelerated wound, burn healing and skin graft acceptance for small and large animals and humans and for the avoidance of wound ischemia.

Another embodiment of the invention is the use of the fluid for lavage purposes post surgery to increase healing and reduce pathogenic anaerobic bacteria populations.

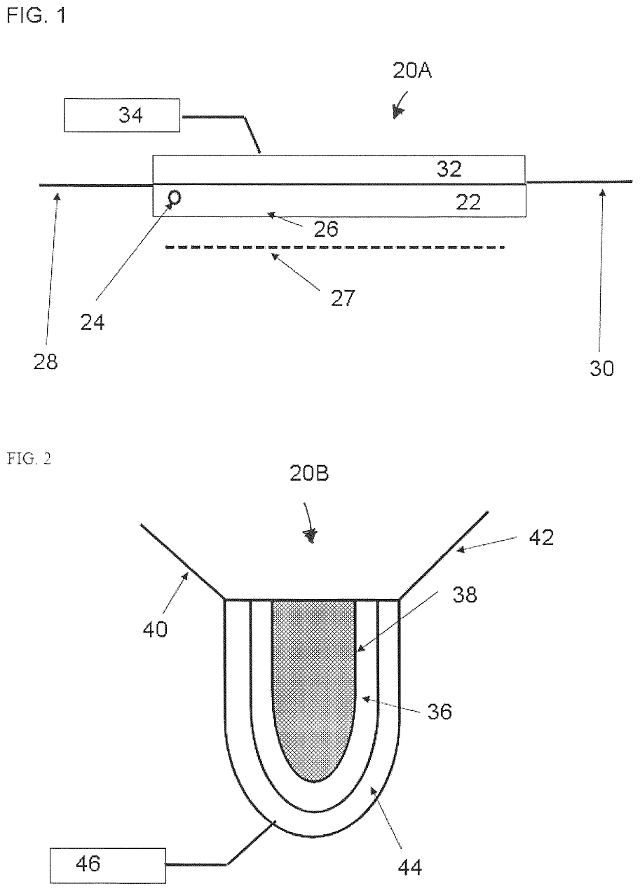

Another embodiment involves the transdermal introduction of oxygen into an organism through the use of a pressurable cuff having a chamber into which the fluid is introduced, the chamber having a semi-permeable membrane which is positioned against the skin of the organism under pressure, all as shown in FIG. 1

Another embodiment of the invention is the introduction of the fluid via a neoprene udder bag as shown in FIG. 2 for the treatment of bovine mastitis, the udder bag functioning in a manner similarly to that of the cuff but for the form factor.

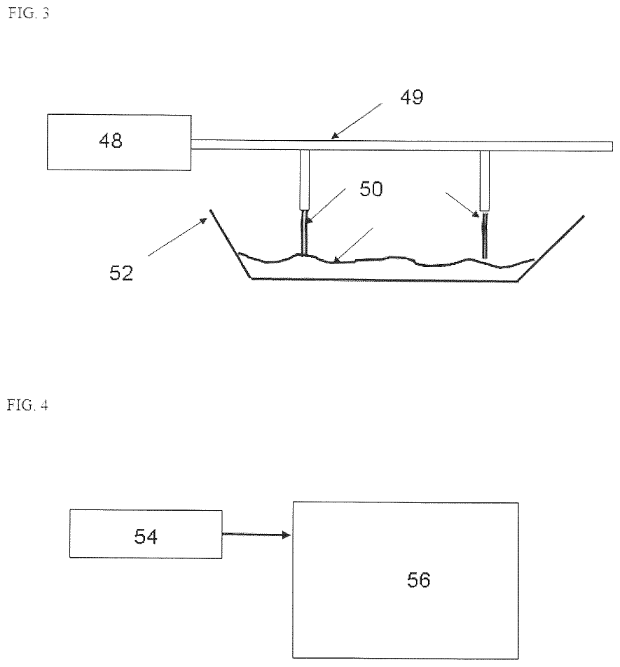

Another embodiment of the invention is the use of the fluid, in aqueous solution form, comprising vaccine in a drinking water supply for poultry or swine, as shown in FIG. 3.

Another embodiment of the invention is the use of the fluid in the form of an aqueous solution comprising vaccine in a tank containing live shrimp.

Another embodiment of the invention is the use of the fluid in the form of an aqueous solution comprising antibiotic in a drinking water supply for poultry, swine or cattle, for enhanced drug uptake.

Another embodiment of the invention is the use of the fluid in the form of an aqueous solution comprising antibiotic in a tank containing live shrimp for enhanced drug update.

Another embodiment of the invention is the introduction of the fluid into the header tank or well-feeder line in a milk cattle barn for the improvement of nutrient absorption, immune response and milk production.

Another embodiment of the invention is the introduction of the fluid in the form of water into a bath or whirlpool, as shown in FIG. 4, for a diabetic to prevent ischemia.

Another embodiment of the invention is the introduction of the fluid in the form of water into a bath or whirlpool for athletic recovery.

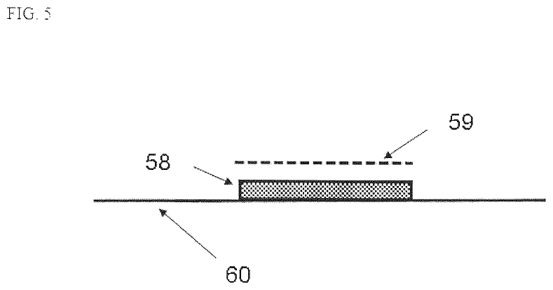

Yet another embodiment of the invention is the introduction of the fluid via a bandage, as shown in FIG. 5, the bandage functioning in a manner similarly to that of the cuff but for the form factor.

Whereas specific embodiments are shown and described, it will be apparent that variations are possible. Without limitation in this regard, devices other than that shown in U.S. Pat. No. 6,209,855 can be used to infuse the oxygen to the desired levels while maintaining total gas pressure. Accordingly the invention should be understood to be limited only by the accompanying claims, purposively construed.

1. Use of a super-oxygenated fluid selected from water, an aqueous solution and a gel, wherein the fluid is introduced to a living organism via any one of the following routes: transcutaneous, intradermal, subcutaneous, intraperitoneal and oral.

2. Use according to claim 1, wherein the level of dissolved oxygen is greater than 185% of saturation at normal total gas pressure.

3. According to another aspect of the invention, the fluid can be oxygenated using the device described in U.S. Pat. No. 6209855.

4. According to another aspect, the fluid can be used in the healing of a wound, wound pO₂ can be monitored in real-time and the fluid can be introduced in response to wound site pO₂.
